(19) United States
(12) Patent Application Publication
(10) Pub. No.: US 2006/0040872 A1
(43) Pub. Date: Feb. 23, 2006

(54) Calcium channel inhibitor

(75) Inventors: Katsuhiro Osajima, Yawatahama-shi (JP); Kiyoshi Matsumoto, Chikushno-shi (JP); Toshiro Matsui, Fukuoka-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: SENMI EKISU CO., LTD, Ohzu-shi (JP); KYUSYU UNIVERSITY, Fukuoka-shi (JP)

(21) Appl. No.: 11/058,162

(22) Filed: Feb. 16, 2005

(30) Foreign Application Priority Data
Aug. 18, 2004 (JP) 2004-238461

(51) Int. Cl. A61K 38/04; C12P 21/06

(52) U.S. Cl. 514/19; 435/68.1

(57) ABSTRACT

The present invention relates to an agent which inhibits or suppresses calcium channel where dipeptide Val-Tyr (VY) or a substance containing the same is an effective ingredient. Examples of the substance containing VY are hydrolysate (sardine peptide) of fish meat of sardine, etc., and VY or VY-containing substance is also able to be used as food/beverage in addition to pharmaceuticals. In accordance with the present invention, it has been clarified that, in reduction of blood pressure, VY has two actions of calcium channel inhibition in addition to ACE inhibition.

### TECHNICAL FIELD OF THE INVENTION

The present invention relates to a calcium channel inhibitor and, more particularly, it relates to a novel and useful calcium channel inhibitor where a specific dipeptide and/or a substance containing the same are/is an effective ingredient(s). Further, the present invention relates to a method for inhibiting calcium channel in a mammal including human in need thereof, which comprises administering said dipeptide and/or said substance, i.e., a material comprising said dipeptide, to the mammal in an effective amount to inhibit calcium channel, or which comprises ingesting a food comprising said dipeptide in an effective amount to inhibit calcium channel.

### BACKGROUND ART

The present inventor Katsuhiro OSAJIMA et al. had succeeded previously in developing a novel peptide α-1000 (peptide mixture) having an ACE (angiotensin I-converting enzyme) inhibiting activity by such a process that fish meat is subjected to a thermal denaturation treatment, autolytic enzyme is inactivated, hydrolysis is conducted using protease, the enzyme is inactivated and a separation treatment is conducted, and have already obtained a patent right (refer to the Patent Document 1).

On the other hand, the present invention has been achieved on a useful and new finding that natural peptide or, particularly, a specific peptide (Val-Tyr) derived from nature has a calcium channel inhibiting action although it has not been known yet at all that the dipeptide has such an excellent physiological action.
Patent Document 1: Japanese Patent No. 3,117,779

### DISCLOSURE OF THE INVENTION

### PROBLEMS THAT THE INVENTION IS TO SOLVE

The present invention has been conducted with an object of development of pharmaceuticals and functional foods derived from nature especially from a viewpoint of safety and has been conducted with an object of newly developing a novel peptide having an excellent physiological action by paying attention again to an excellent physiological action of peptide α-1000 which is a peptide mixture derived from fish meat and previously developed successfully by the present inventor Katsuhiro OSAJIMA et al.

### BRIEF DESCRIPTION OF THE DRAWINGS

FIG. 1 shows an infrared absorption spectrum of peptide α-1000.

FIG. 2 shows an ultraviolet absorption spectrum of peptide α-1000.

FIG. 3 shows a molecular weight distribution of peptide Y-2 by gel filtration.

FIG. 4 shows an infrared absorption spectrum of peptide Y-2.

FIG. 5 shows an ultraviolet absorption spectrum of peptide Y-2.

FIG. 6 shows molecular weight of peptide SY.

FIG. 7 shows an infrared absorption spectrum of peptide SY.

FIG. 8 shows an ultraviolet absorption spectrum of peptide SY.

FIG. 9 shows an infrared absorption spectrum of peptide SY-MD.

FIG. 10 shows an ultraviolet absorption spectrum of peptide SY-MD.

FIG. 11 is a graph of elution patterns of peptides SY and SY-MD.

FIG. 12 shows the effect of addition of VY in the presence of 5% FBS.

FIG. 13 shows the result of toxicity test of VY.

FIG. 14 shows the effect of VY on Ang II stimulation.

FIG. 15 shows the dependency of VY concentration to the growth of VSMC by Ang II stimulation.

FIG. 16 shows the influence of ACE inhibitor on Ang II stimulation.

FIG. 17 shows the influence of VY and saralasin (Ang II antagonist) on Ang II stimulation.

FIG. 18 shows the influence of VY on Bay K 8644 (Ca²⁺ channel agonist).

FIG. 19 shows the dependency of VY concentration to Bay K 8644 stimulation.

### MEANS FOR SOLVING THE PROBLEMS

In order to achieve the above-mentioned objects, the present inventors have investigated from various views, paid their attention to peptides derived from fish meat, conducted studies for physiologically active function thereof and found as a result that peptides derived from fish meat have an excellent suppressive action to hypertension. They have also confirmed that such an action is mostly due to inhibition to angiotensin I-converting enzyme (ACE) which catalyzes production of angiotensin II (Ang II) showing a vasoconstrictive action from angiotensin I (Ang I). During the course of the study, a phenomenon which was not able to be explained only by an ACE suppressive action was noted in an in vivo test.

In view of the above, the present inventors have obtained a new idea that a hypotensive mechanism other than an ACE inhibitive action may be due to peptide derived from fish meat and conducted investigations from various views. As a result, they paid their attention for the first time to an inhibitive or suppressive action on calcium channel as a hypotensive mechanism other than an ACE inhibitive action by peptides derived from fish meat. Although calcium is necessary for the growth of cells, flexibility of blood vessel lowers when calcium is excessively incorporated into blood vessel cells via calcium channel whereupon blood pressure rises and, therefore, it is necessary to appropriately close the calcium channel so that absorption of calcium is prevented (inhibited).

The present inventors have fractionated the peptides derived from fish meat and investigated each of the obtained various fractionated factions using normal human aortic vessel smooth muscle cells (VSMC) for the purpose of clarification of physiological activity thereof and, as a result, they have firstly found that dipeptide Val-Tyr (valyl-tyrosine; hereinafter, it may be referred to VY) has an action of inhibiting or suppressing calcium channel.

In addition, investigations have been conducted not only for the isolated VY but also for screening of substances containing VY, separation of compositions containing a high amount of VY and method for preparing the same and the present inventors have firstly confirmed that those VY-containing substances also have an excellent inhibitive (or suppressive) action on calcium channel. As a result of various studies on the basis of such useful and new findings, the present invention has been at last achieved.

Thus, the present invention is based on the fact that a new physiological action (new hypotensive action which is different from ACE-inhibitive action) of a dipeptide VY, which is an inhibitive (or suppressive) action on calcium channel has been firstly found and it relates to a pharmaceutical or food/beverage to inhibit (or suppress) or prevent calcium channel, VY and/or a VY-containing substance being an effective ingredient(s). Such pharmaceutical and/or food/beverage are/is useful, for example, for lowering the blood pressure or for suppressing the rise of the blood pressure in a preventive manner.

In the present invention, dipeptide VY is used as an effective ingredient and, with regard to VY, that which is purified and isolated is used and, in addition, various kinds of compositions containing VY, particularly, substances derived from natural substance may also be used. With regard to such substances, a processed product of fish meat, for example, has been found to be suitable as a result of the studies by the present inventors. A chemically-synthesized VY may be used.

The processed product described here means a product prepared by one or more process (es) which is/are commonly used for separation and purification of natural substances such as amino acids, peptides and proteins and, with regard to the process, the following processes may be exemplified. They are dialysis, enzymatic hydrolysis, acidic hydrolysis, defatting, ion-exchange using resin, chromatography and others.

In the present invention, it is also possible to use, as an effective ingredient, a VY-containing substance derived from a natural material. Examples of such a substance are processed product of wheat germ and processed product of fish meat, and a peptide mixture derived from wheat germ and a peptide mixture derived from fish meat such as α-1000, Y-2, SY and SY-MD are advantageously used. If desired, separation and purification means such as chromatographic treatment may be repeated or appropriately combined whereby it is possible that VY concentration is increased or VY fraction is fractionated.

As an example of a VY-containing substance, a processed product of fish meat will be illustrated as hereunder.

As a result of continued studies from various views, the present inventors have firstly found that VY is contained in a high concentration in a fraction which is prepared in such a manner that peptide α-1000 previously developed by the present inventor Katsuhiro OSAJIMA et al., which is a peptide mixture derived from fish meat, is subjected to a hydrophobic adsorption resin ODS column and then eluted with 10 v/v % aqueous solution of ethanol and, as a result of intensive studies, they have succeeded in developing a processed product of fish meat (peptide Y-2) containing a high content of VY. They have also confirmed that VY is contained in a high content in processed products of fish meat (peptide SY, peptide SY-MD, etc.) according to other processes. Thus, they have succeeded in completing inventions from the view of peptides derived from fish meat.

For example, peptide Y-2 is able to be prepared as follows With regard to a starting liquid or a material for peptide Y-2, a unpurified solution containing peptide derived from fish meat is used. This solution is treated with a hydrophobic absorptive resin, elution is conducted with a 5 to 20 v/v %, preferably a 8 to 17 v/v % or, still more preferably about 15 v/v % aqueous solution of ethanol to give peptide Y-2. It is also possible that, before eluting with an aqueous solution of ethanol, elution is conducted with water followed by eluting with an aqueous solution of ethanol. The eluted fraction prepared as such contains VY and the fraction (peptide Y-2) is able to be advantageously used as an effective ingredient in the present invention.

As to a material or a source for SY and SY-MD in addition to peptide Y-2, in other words, as to a starting peptide liquid, it is possible, for example, to use an aqueous solution of peptide α-1000.

Peptide α-1000 is able to be prepared by such a manner that fish meat is subjected to a thermal denaturation and hydrolyzed by treating with neutral or alkaline protease, then the enzyme is inactivated by a common method such as heating and a separation treatment is conducted. Details are mentioned as follows.

Peptide α-1000 is prepared using fish and/or shellfish as raw material and, for example, it may be prepared according to Japanese Patent No. 3,117,779. Firstly, fish and/or shellfish are/is processed in a meat collector, a deboner or the like to separate their meat. It is desirable that the raw material is as fresh as possible. The separated meat is ground and divided into plural lumps of ground fish meat weighing about 10 kg each and, although those lumps may be subjected to the next treatment, they may be rapidly frozen with a spray of cold air of from −20° C. to −50° C. or, for example, about −30° C. and stored at −20° C. to −25° C. and, if necessary, that may be used appropriately.

The fish and shellfish usable herein include, for example, fishes with red flesh such as sardine, saurel, tuna, bonito, saury and mackerel; fishes with white flesh such as flounder, sea bream, sillaginoid, gizzard shad, cod, herring and yellowtails; cartilaginous fishes such as shark and ray; freshwater fishes such as pound smelt, carp, char and yamame (a kind of trout); deep-sea fishes such as granulose and anglerfish; as well as lobster, prawn, shrimp, crab, octopus, opossum shrimp, etc.

The fish and/or shellfish meat collected as such is ground with a meat grinder or the like, to which is added water of from ½ to 20 times, but preferably from 1 to 10 times by weight the meat. Then this is heated thereby to inactivate the autolytic enzyme existing therein and also to kill bacteria, simultaneously the meat protein being thereby thermally denatured so as to increase the subsequent enzymatic reaction efficiency. For heating it, employable is any condition capable of producing the intended results and, for example, it may be heated in the temperature range of not lower than 65° C. for a period of from 2 to 60 minutes or preferably not lower than 80° C. for a period of from 5 to 30 minutes.

Next, an alkaline agent such as aqueous ammonia or an aqueous solution of sodium (or potassium) hydroxide is added thereto so as to make the meat have a pH value suitable to protease with which the meat is to be processed. (For example, for alkaline protease, the pH may be at least 7.5 or, preferably, at least 8). The meat is heated at a temperature also suitable to the protease (for example, at 20 to 65° C. although that depends on the type of the protease to be used; for example, for alkaline protease, the meat is heated at 35 to 60° C. or, preferably, 40 to 55° C.) and the meat is processed with protease for 30 minutes to 30 hours (for example, in the case of an alkaline protease, for 30 minutes to 25 hours or, preferably, 1 to 17 hours).

With regard to the protease, any enzyme may be used so far as it is capable of degrading protein in neutral or alkaline condition either solely or jointly. As to its origin, protease may be derived not only from animals and vegetables but also from microorganisms and it covers a broad range of various proteases including, for example, pepsin, renin, trypsin, chymotrypsin, papain, bromelain as well as bacterial proteases, filamentous proteases, actinomycelial proteases, etc. Usually, those enzymes which are available in the market may be used but, depending on their applications and upon necessity, also usable are non-purified enzymes as well as solid or liquid, enzyme-containing substances such as enzyme-containing cultures and koji (malted rice or malt). The amount of the enzyme to be added may be about 0.1 w/w % to 5 w/w %.

If desired, the thus-treated meat is then neutralized and heated at not lower than 70° C. (preferably, at 80° C.) for 2 to 60 minutes (preferably, 5 to 30 minutes) so as to inactivate the enzyme used and to facilitate the separation to be carried out later. The meat having subjected to the heat treatment for enzyme inactivation as such is then passed through a vibro-screen or the like to remove coarse impurities therefrom, then optionally passed through Jector and finally subjected to ultra-centrifugation to remove floating impurities and precipitated impurities therefrom.

After that, the obtained product is filtered using a filter aid such as diatomaceous earthy (such as Celite) and the obtained filtrate is treated with activated carbon so as to deodorize, decolor and purify it (for which the amount of activated carbon to be used may be from 0.05 to 20 w/v % or, preferably, from 0.1 to 10 w/v % and the treatment with activated carbon may be effected at 20 to 65° C. or, preferably, 25 to 60° C. for 15 minutes to 4 hours or, preferably, 30 minutes to 2 hours.

The purified product is then concentrated in any ordinary manner, for example, under reduced pressure (at 0 to 50° C. to an extent of about 30 Bx). If desired, this is again subjected to (ultra)centrifugation or filtration to obtain a peptide solution. The thus-obtained peptide solution is sterilized (through UHTST or in any other ordinary manner) and filled into containers to give a product (α-1000 (liquid)). If desired, this may be further concentrated or may be even diluted, or may be powdered in any ordinary manner such as spray-drying and freeze-drying into a powdery product of about 60-mesh and the powder may be packed in bags or any other container to give a product (α-1000 (powder)). These products are stored in refrigerators or freezers in the case of liquid or stored in a dry, cool and dark place in the case of powder.

The peptide mixture in the liquid, paste or powder from prepared as such is peptide α-1000.

The physico-chemical properties of peptide α-1000 (spray-dried powder) are as shown below.

Physico-Chemical Properties of Peptide α-1000 (Powder)

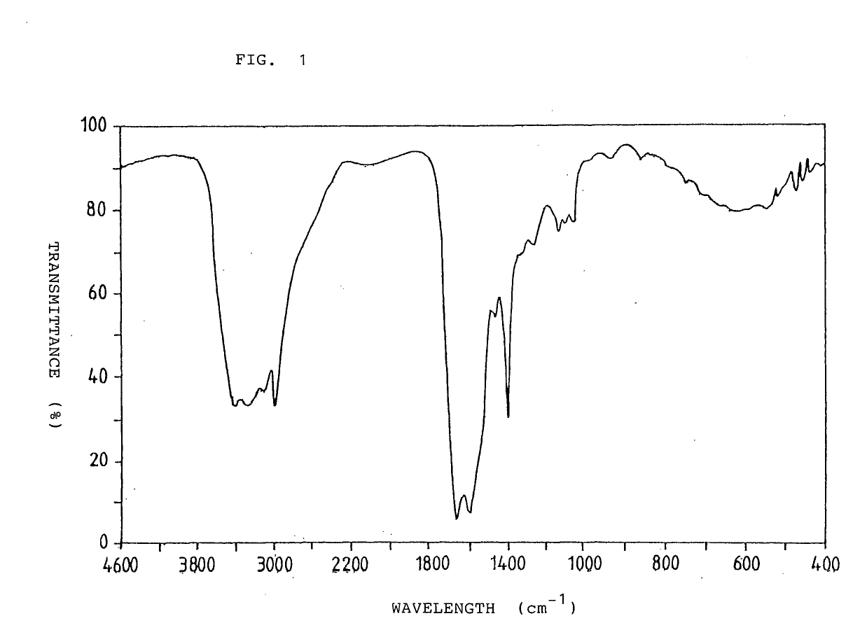

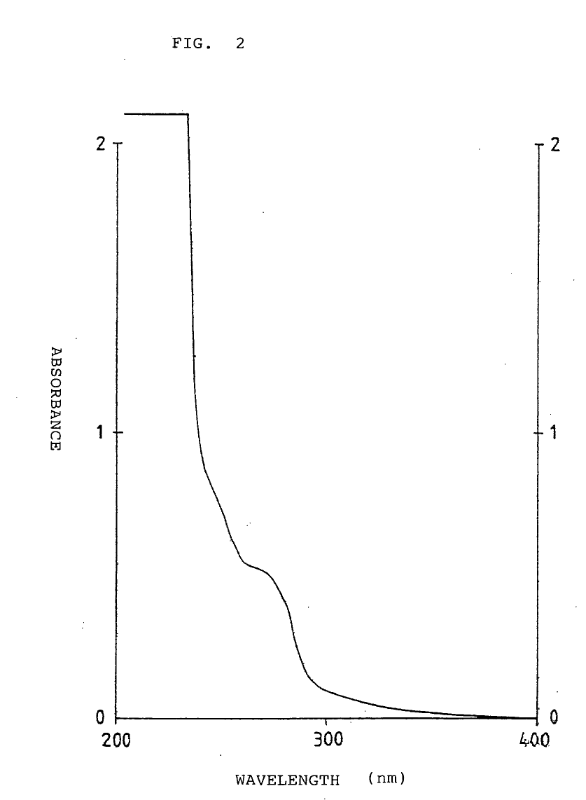

(A) Molecular weight: 200 to 10,000 (as measured by Sephadex G-25 column chromatography);
(B) Melting point: Colored at 119° C. (decomposition point);
(C) Specific rotatory power: [α]_{D}²⁰=−22°;
(D) Solubility in solvents: It is easily soluble in water but rarely soluble in ethanol, acetone and hexane;
(E) Chemical differentiation in acidic, neutral or basic character: Neutral; pH of from 6.0 to 8.0 (10 w/v % solution);
(F) Appearance and constituent components: It is white powder comprising 5.14 w/w % of water (by a vacuum heating and drying method), 87.5 w/w % of protein (by a Kjeldahl method with a nitrogen/protein conversion coefficient of 6.25), 0 w/w % of lipid (by a Soxhlet extraction method) and 5.0 w/w % of ash (by a direct ashing method);
(G) Characteristics: It is a peptide mixture derived from fish meat and obtained by inactivating an autolytic enzyme by heating followed by hydrolyzing with protease;
It contains dipeptide Val-Tyr and has an action of inhibition (or suppression) on calcium channel;
(H) Infrared absorption spectrum: FIG. 1;
(I) Ultraviolet absorption spectrum: FIG. 2; and

(J) Amino acid composition: As shown below:

TABLE 1
Amino Acid Composition of Peptide α-1000 (Powder)

| Items for Analytical Test | |
|---|---|
| Total Amino Acid | Result (%) |
| Arginine | 3.34 |
| Lysine | 6.86 |
| Histidine | 3.34 |
| Phenylalanine | 2.33 |
| Tyrosine | 2.01 |
| Leucine | 6.35 |
| Isoleucine | 3.27 |
| Methionine | 2.26 |
| Valine | 4.16 |
| Alanine | 5.17 |
| Glycine | 3.59 |
| Proline | 2.15 |
| Glutamic acid | 12.35 |
| Serine | 3.30 |
| Threonine | 3.70 |
| Aspartic acid | 8.36 |
| Tryptophan | 0.32 |
| Cystine | 0.47 |
| Total Amount | 73.33 |

Analytical method: Automatic analysis of amino acids (except that cystine was oxidized with formic peracid followed by hydrolyzing with hydrochloric acid and that tryptophan was analyzed by means of high performance liquid chromatograph)

Although peptide α-1000 thus-prepared may be utilized as an effective ingredient in the present invention, it may be further processed such as that, for example, in the case of liquid, it is directly or, in the case of powder, after water is added thereto, it is passed through a column of hydrophobic adsorptive resin such as ODS, eluted with water and then eluted with a 5 to 20 v/v %, preferably 11 to 19 v/v % or, more preferably 13 to 18 v/v % aqueous solution of ethanol, thereby peptide Y-2 being obtained. Incidentally, with regard to the resin, any resin may be used so far as it is a hydrophobic adsorptive resin and commercially available resins being mentioned already may be able to be appropriately used.

In the Y-2 fraction (i.e., peptide Y-2) which is a purified peptide mixture thus-obtained from fish meat, a lot of VY is contained and, in fact, it was confirmed as a result of analysis by a high-performance liquid chromatography that about 150 mg/100 g of VY was contained in the Y-2 fraction (i.e., a sardine peptide mixture Y-2 which is an enzymatically decomposed and purified product derived from meat of sardine) which was prepared in such a manner that sardine meat was processed with 0.7 w/v % alkalase for 17.5 hours, the resulting hydrolysate was subjected to an ODS column, and the latter half of the fraction eluted with water and the fraction eluted with 15 v/v % ethanol were combined.

The physico-chemical properties of peptide Y-2 which is to be used as an effective ingredient in the present invention are as follows.

Physico-Chemical Properties of the Peptide Y-2

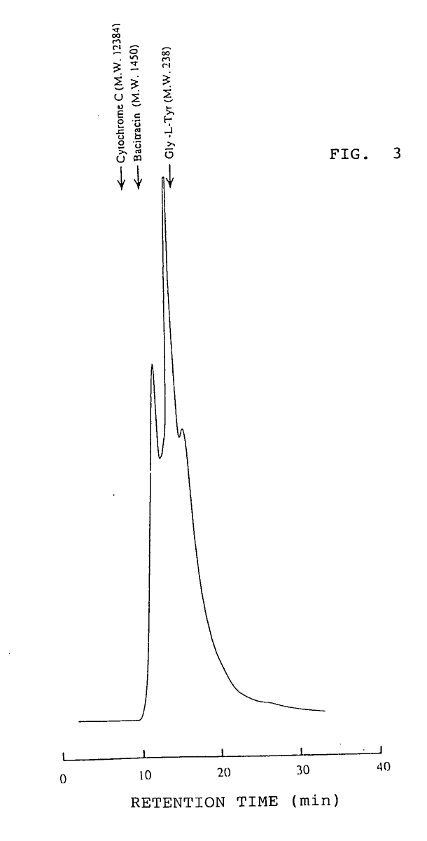

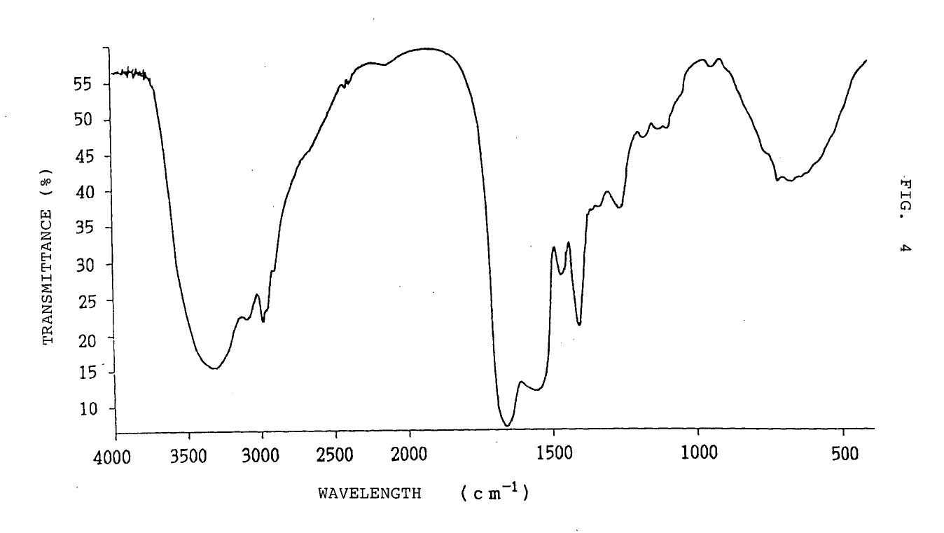

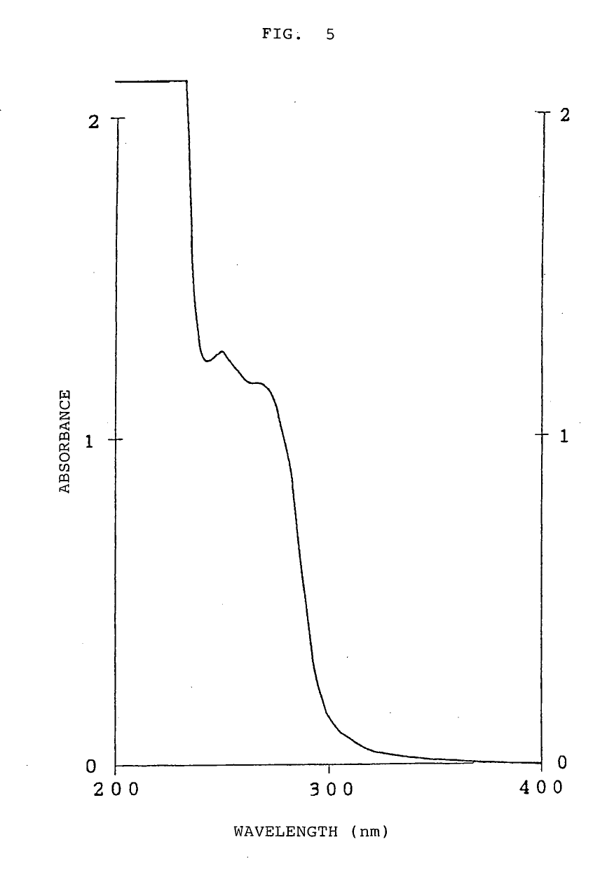

(A) Molecular weight: 200 to 10,000 (as measured by high-performance liquid chromatography using ASAHIPAK GS-320, Asahikasei Co.) (FIG. 3);
(B) Melting point: It is colored and decomposed at 138° C.;
(C) Specific rotatory power: [α]_{D}²⁰=−40°;
(D) Solubility in solvents: It is easily soluble in water but rarely soluble in ethanol, acetone and hexane;
(E) Chemical differentiation in acidic, neutral or basic character: Neutral; pH of from 5.0 to 8.0 (10 w/v % solution);
(F) Appearance of the substance: It is white to light yellow powder;
(G) Constituent components: It comprises 2.72 w/w % of water (by a heating and drying method under ordinary pressure), 87.25 w/w % of protein (by a Kjeldahl method with a nitrogen/protein conversion coefficient of 6.25), 0 w/w % of lipid (by a Soxhlet extraction method) and 0.20 w/w % of ash (by a direct ashing method);
(H) Physiological properties: It contains dipeptide Val-Tyr and has an inhibitive or suppressing action to calcium channel;
(I) Infrared absorption spectrum: FIG. 4;
(J) Ultraviolet absorption spectrum: FIG. 5; and

(K) Amino acid composition: As shown in the following Table 2; the analytic method was according to an automatic analysis of amino acids (Shimadzu LC-6A system):

TABLE 2
Amino Acid Composition of Peptide Y-2

| Amino Acids | Result (%) |
|---|---|
| Aspartic acid | 10.97 |
| Threonine | 4.10 |
| Serine | 2.90 |
| Glutamic acid | 12.52 |
| Glycine | 4.91 |
| Alanine | 5.06 |
| Valine | 6.20 |
| Methionine | 2.55 |
| Isoleucine | 5.55 |
| Leucine | 9.47 |
| Tyrosine | 2.96 |
| Phenylalanine | 4.75 |
| Histidine | 2.83 |
| Lysine | 10.07 |
| Arginine | 7.50 |

Further, the present inventor Katsuhiro OSAJIKA et al. have paid their attention to the usefulness of the above-mentioned peptide Y-2 again and studied a peptide mixture (such as peptide α-1000) prepared by the treatment of fish meat with protease and, when a peptide mixture derived from fish meat was treated with a hydrophobic adsorptive resin (such as ODS resin) and subjected to a three-step elution comprising elution with water, elution with aqueous ethanol and elution with water, they have found a useful finding that VY in the fish meat peptide mixture was recovered in large quantities in apart (especially a latter fraction) of the fraction in the first elution with water (1), in the fraction eluted with an aqueous solution (especially a 11 to 18 v/v % solution) of ethanol (ethanol elution) and in the final fraction eluted with water (2) (water elution (2)). The part of the fraction eluted with water (1), the fraction eluted with the aqueous solution and the fraction eluted with water (2) are mixed to give peptide SY.

As such, a mixture of the latter fraction at the first elution with water (1), the fraction eluted the with 11 to 18 v/v % aqueous solution of ethanol (ethanol elution) and in the final fraction eluted with water (2) not only contains a high amount of VY but also has little bitter taste, shows an excellent taste and is excellent in stability whereby it has been confirmed to be an entirely novel functional peptide mixture, identified as a novel peptide mixture and named peptide SY.

Further, in said invention, when only the above fraction eluted with the 11 to 18 v/v % aqueous solution of ethanol was isolated and tested, a new peptide mixture containing Na in an amount of as very small as about 0.1 to 0.2 w/w % (in this peptide SY, Na is about 1 to 3 w/w %) was found and, therefore, that fraction was named peptide SY-MD.

In said invention, peptide α-1000 was used as a starting material and studies were conducted for a purpose of continuous recovery of a peptide mixture containing Val-Tyr as much as possible.

As a result, it was found that, when α-1000 was adsorbed with ODS resin, water was added, a part of fraction (a fraction in the latter stage) eluted with water (1) is prepared and, after that, aqueous solution of ethanol was added thereto continuously to prepare a fraction which was eluted with the aqueous solution of ethanol, the ethanol concentration was appropriately to be 11 to 18 v/v % or, preferably, 14 to 16 v/v % because a part of water used for elution with water (1) still remained.

Furthermore, in the preparation of peptide SY, measurements and monitorings were carried out on elution time, salt concentration, Bx and UV absorption at 280 nm of wavelength for determining the initiation point to obtain the latter fraction of the elution with water (1), the end point of said elution (i.e., the initiation point of the elution with the aqueous solution of ethanol), the end point of said elution with the aqueous solution of ethanol (i.e., the initiation point of the elution with water (2)) and the end point of the elution with water (2), thereby a series of continuous systems to produce peptide SY being established. On the basis of such useful findings, further studies were conducted and, at last, the present invention has been achieved.

Thus, the present invention relates to an inhibitive or suppressive agent for calcium channel, characterized in that, peptide SY or peptide SY-MD containing dipeptide VY is an effective ingredient and the physico-chemical properties thereof are as follows.

Physico-Chemical Properties of Peptide SY

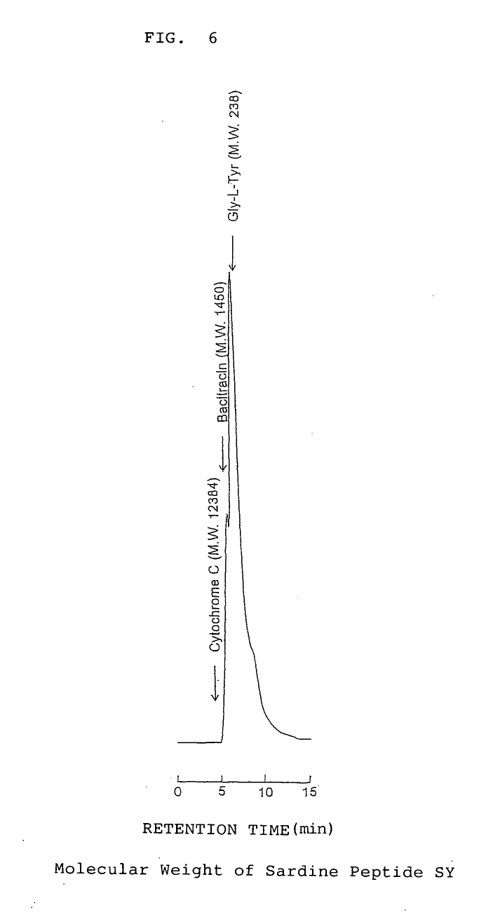

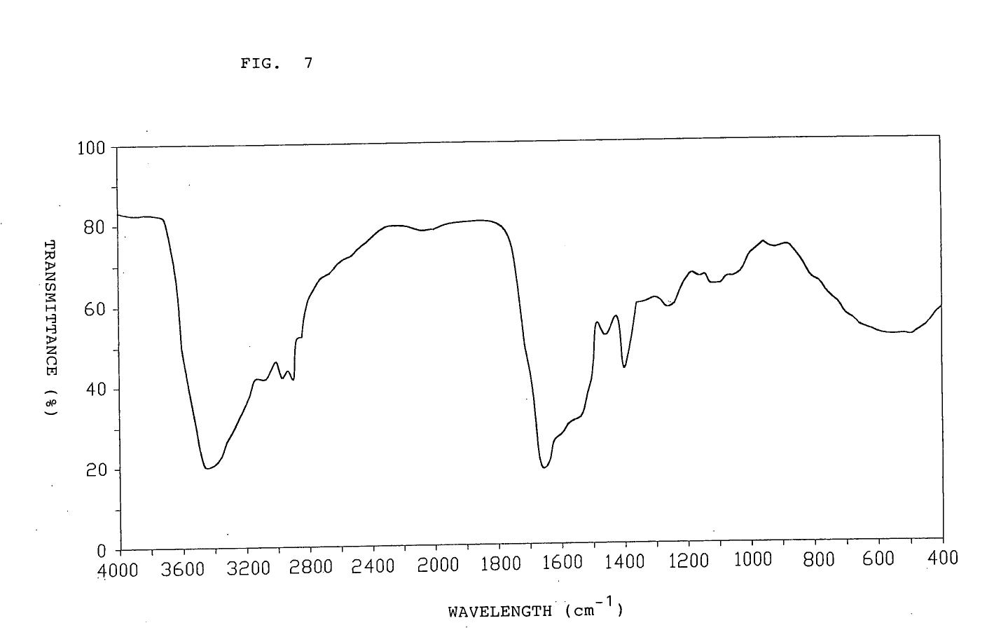

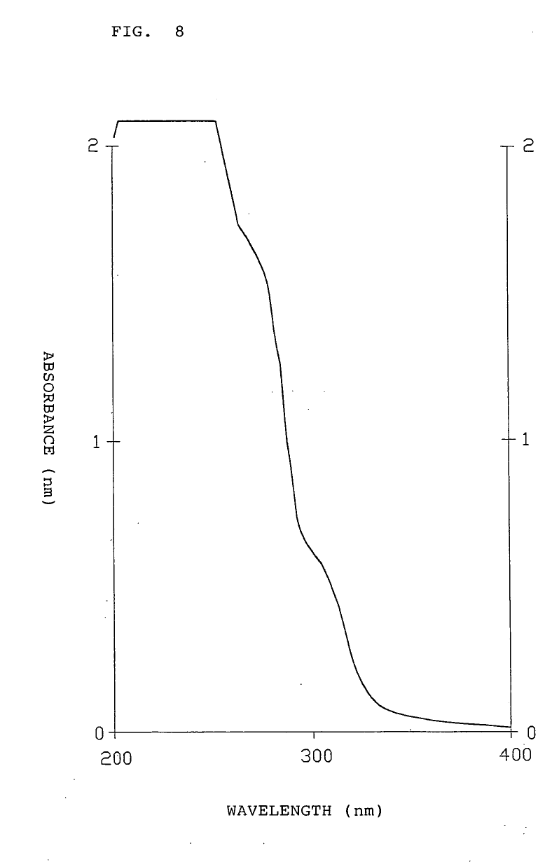

(A) Molecular weight: 200 to 10,000 (as measured by high-performance liquid chromatography using ASAHIPAK GS-320, Asahikasei Co.); FIG. 6;
(B) Melting point: It is colored and decomposed at 138 ±3° C.;
(C) Solubility in solvents: It is easily soluble in water but rarely soluble in ethanol, acetone and hexane;
(D) Appearance and property: It is white to light yellow powder;
(E) Liquid property (pH): 4.0 to 6.0;
(F) Constituent components: It comprises 1 to 5 w/w % of water (by a heating and drying method under ordinary pressure), 84 to 94 w/w % of protein (by a micro-Kjeldahl method), not more than 0.5 w/w % of lipid (by a Soxhlet extraction method), 4±2 w/w % of ash (by a direct ashing method) and 1 to 3 w/w % of Na (by an atomic absorption spectrophotometry);
(G) Physiological properties: It contains dipeptide Val-Tyr and has an inhibitive action to the growth of blood vessel smooth muscle cells;
(H) Infrared absorption spectrum: FIG. 7;
(I) Ultraviolet absorption spectrum: FIG. 8;
(J) Specific rotatory power: [α]_{D}²⁰=−40° to −51°; and

(K) Main amino acid composition: As shown in the following Table 3; the analytic method was according to an automatic analysis of amino acids (Shimadzu LC-6A system):

TABLE 3
Amino Acid-Composition of Peptide SY

| Amino Acids | Results (%) |
|---|---|
| Aspartic acid | 8.0 to 9.2 |
| Glutamic acid | 9.5 to 12.0 |
| Valine | 4.5 to 5.5 |
| Methionine | 2.5 to 3.8 |
| Isoleucine | 4.5 to 5.2 |
| Leucine | 7.3 to 8.5 |
| Tyrosine | 3.4 to 4.8 |
| Phenylalanine | 4.5 to 5.5 |
| Histidine | 3.0 to 3.8 |
| Lysine | 6.5 to 7.8 |
| Arginine | 5.0 to 6.0 |

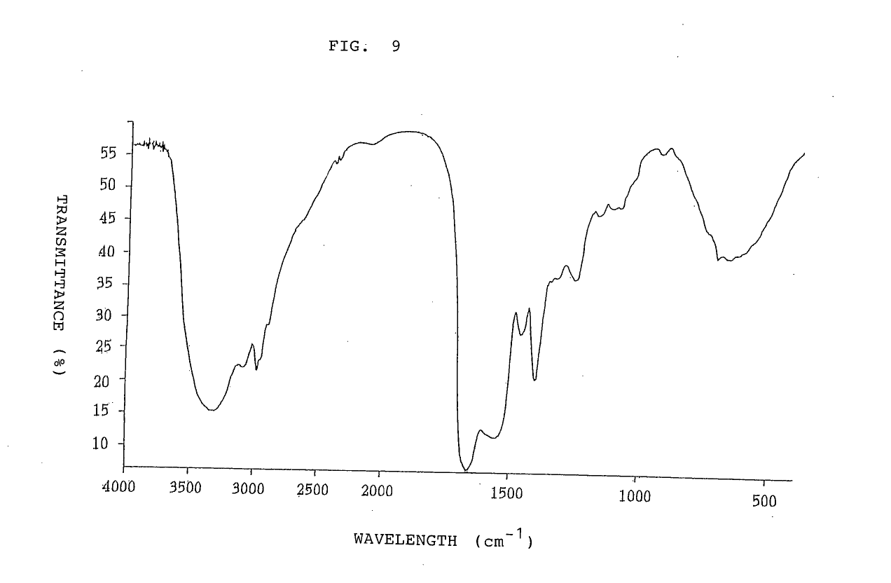

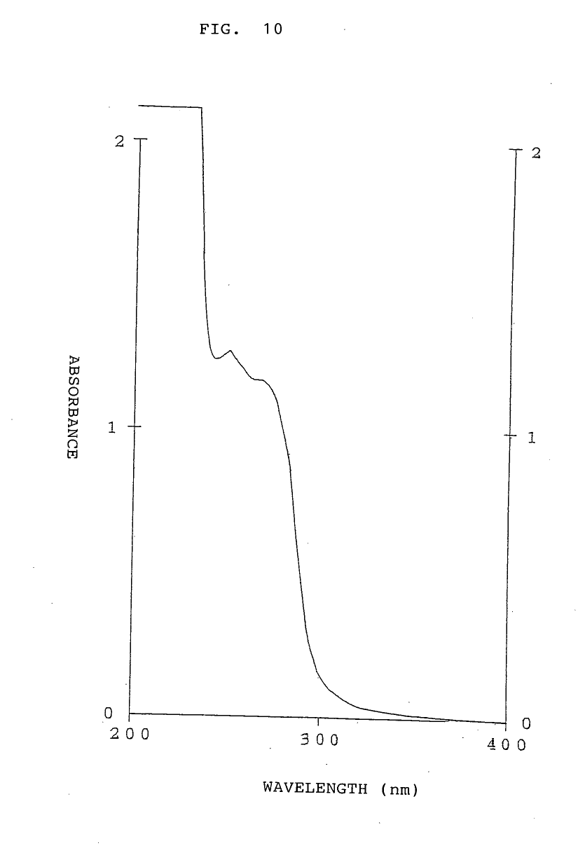

Physico-Chemical Properties of Peptide SY-MD
(A) Molecular weight: 200 to 10,000;
(B) Melting point: It is colored and decomposed at 138 ±3° C.;
(C) Solubility in solvents: It is easily soluble in water but rarely soluble in ethanol, acetone and hexane;
(D) Appearance and property: It is white to light yellow powder;
(E) Liquid property (pH): 4.0 to 6.0;
(F) Constituent components: It comprises 2 to 6 w/w % of water (by a heating and drying method under ordinary pressure), 90 to 98 w/w % of protein (by a micro-Kjeldahl method), 0.5 w/w % of lipid (by a Soxhlet extraction method), 3.0 w/w % of ash (by a direct ashing method) and 0.1 to 0.2 w/w % of Na (by an atomic absorption spectrophotometry);
(G) Physiological properties: It contains dipeptide Val-Tyr and has an inhibitive action to the growth of blood vessel smooth muscle cells;
(H) Infrared absorption spectrum: FIG. 9; and
(I) Ultraviolet absorption spectrum: FIG. 10.

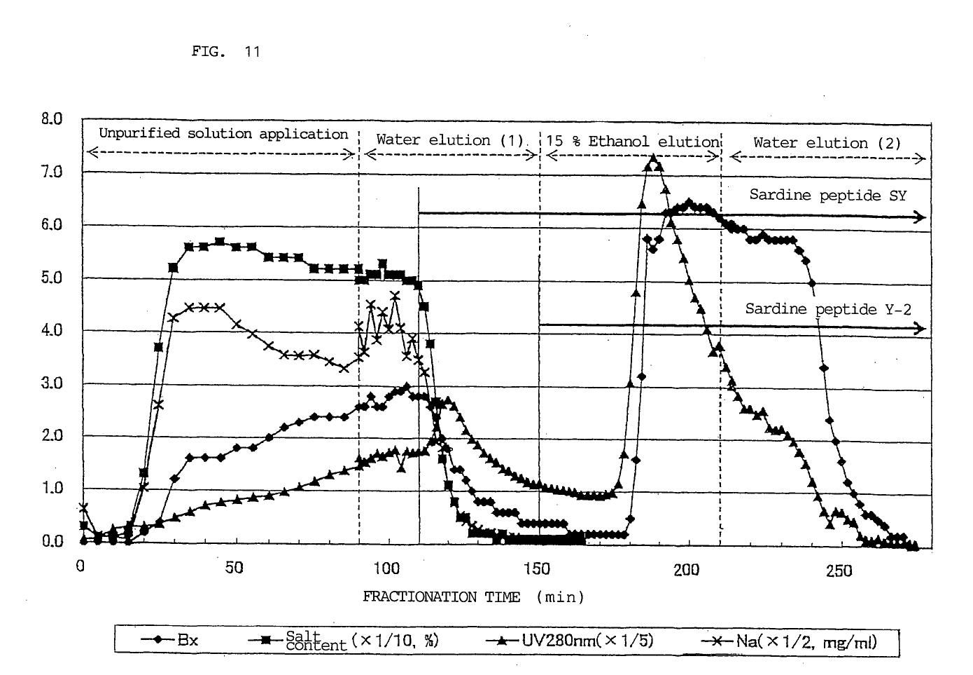

Peptide SY is able to be produced as follows. That is, a unpurified solution containing peptide, in other words, an enzymatically processed substance of fish meat which is to be a starting material for peptide SY (e.g., peptide α-1000), as it is in the case of liquid or after being added water thereto in the case of powder, is applied into a column of hydrophobic adsorptive resin such as an ODS resin, thereby “Unpurified solution application” of FIG. 11 being conducted and the production process being started.

Thus, in eluted patterns exemplified by FIG. 11 which are peptide patterns obtained by fractionation treatment by elution of the unpurified solution containing peptide in making use of the hydrophobic adsorptive resin where the fractionation treatment by elution is conducted in the order of water, aqueous solution of ethanol and water as developers, the latter fraction by eluting with water (1), the fraction by eluting with a 11 to 18 v/v % aqueous ethanol solution (in FIG. 11, elution with 15% ethanol is shown) and the fraction eluted with water (2) obtained by each developer as stipulated below are prepared and mixed to produce peptide SY.

(1) The latter fraction by eluting with water (1): A fraction obtained by using water as an eluent of from a time when a sodium (Na) content of the whole-fraction (peptide-SY) eluted with become 1 to 3 g/100 g to a final collection time of the latter fraction in the water elution (1) when the sodium content becomes substantially 0 g/100 g.

(2) Fraction by eluting with a 11 to 18 v/v % ethanol: A fraction obtained next by using an ethanol aqueous solution having a concentration of a 11 to 18 v/v % as an eluent until an amount of peptide eluted passes a peak and decreases to about the half of the peak (That where only this fraction is isolated is called peptide SY-MD.).

(3) Fraction by eluting with water (2): A fraction thereafter obtained by using water as an eluent until the elution of peptide is completed.

Then, as mentioned above, in (2), only the fraction obtained by using the aqueous ethanol solution, which is eluted until the amount of peptide eluted passes the peak and decreases to about the half of the peak, is collected to give peptide SY-MD containing peptide Val-Tyr. It is also possible to prepare peptide Y-2 from the above (2) or from the above (2) and (1).

Process for the production of those peptide mixtures will be illustrated in detail as hereunder by referring to FIG. 11. That is, peptide SY is able to be produced by collecting the above-mentioned eluted fractions. An example of elution patterns of various eluates is shown in FIG. 11.

As shown in FIG. 11, peptide SY in the present invention is able to be produced in such a manner that, for example, peptide α-1000 is applied (a unpurified solution application) to the hydrophobic adsorptive resin, then elution with water is conducted (Water elution (1)) and the latter fraction of the elution with water (1), the fraction eluted with the 11 to 18 v/v % aqueous ethanol solution and the fraction which is further eluted with water (Water elution (2)) are mixed. Starting point of fractionation (collection) of the peptide SY fraction and times to switch the eluents, etc. may be appropriately decided on the basis of the measurement of at least one of Bx, salt content, UV (absorption at 280 nm) and Na or on the basis of elution times. It is also possible to appropriately monitor these items in real time and perform the determinations by using a computer.

For example, in the elution pattern of FIG. 11, the fractionation starting point of the latter fraction of water elution (1) of peptide SY is able to be determined by measuring the salt content as follows.

i) When collection is started from 0 minute after the initiation of elution with water, the Na content becomes 4 g/100 g or more and, therefore, there are some cases where a high-Na material is resulted and blood pressure rises in the use thereof. Thus, that is not desirable.

ii) When the starting time for collection is 20 minutes after the initiation of the elution with water, the Na content is 1 to 3 g/100 g and that is within an allowable range.

iii) When collection is started after that, the Na content becomes far less but, salt content is too low. Accordingly, guanine contained in peptide SY is apt to separate upon concentration and sediment may be resulted. Thus, that is not desirable.

iv) Accordingly, the time for starting the collection is set at 20 minutes after the initiation of the elution with water and the Na amount becomes approximately 1 to 3 g/100 g.

In addition, the final point for collecting the fraction with water (1) is set at the time when the Na amount becomes substantially 0 g/100 g.

After that, from this time, the 11 to 18 v/v % aqueous solution of ethanol is added in place of water. When the eluted amount of peptide passes the peak and decreases to about one-half of the peak, addition of the aqueous solution of ethanol was stopped and the fraction obtained thereby is used as the fraction eluted with the 11 to 18 v/v % ethanol solution. (When only the fraction eluted with the 11 to 18 v/v % ethanol solution is isolated, the thus-obtained fraction is peptide SY-MD which contains almost no Na.)

The time when the addition of the 11 to 18 v/v % aqueous solution of ethanol is stopped and switched to the addition of water is the starting point for elution with water (2). Said starting point is the time when UV absorption at 280 nm wavelength showing an outstanding decrease of the amount of peptide becomes about one-half of the peak, and the end-point is the time when the UV absorption becomes zero showing a stationary state. The fraction prepared thereby is used as a fraction with water (2).

The thus-obtained latter fraction eluted with water (1), the fraction eluted with the 11 to 18 v/v % ethanol solution and the fraction eluted with water (2) are collected separately or continuously and mixed to give peptide SY in the present invention.

Thus, the whole fraction of from the latter fraction eluted with water (1) to the fraction eluted with water (2) including the fraction eluted with the 11 to 18 v/v % ethanol solution is able to be obtained as peptide SY in the present invention (in FIG. 11, that is shown as sardine peptide SY).

The area shown by “15% Ethanol elution” in FIG. 11 corresponds to “peptide SY-MD”.

Peptides SY, SY-MD, Y-2 and α-1000 (each is a peptide mixture) contain a high concentration of the dipeptide (valyl-tyrosine; Val-Tyr or VY) which has been firstly confirmed by the present inventors as a chief peptide of peptides exhibiting inhibition or suppression action on calcium channel. Particularly, peptide SY-MD does not contain the latter fraction eluted with water (1), and the taste thereof is greatly improved although bitter taste remains a little, and moreover, since it rarely contains Na, it is very useful for persons who must ingest no Na.

Thus, although the portion “Unpurified solution application” has a rich taste, it contains some fish smell derived from the raw material and contains much Na as well. On the contrary, the latter fraction eluted with water (1) has little fish smell derived from the raw material and has a very good taste as well.

Therefore, when the latter fraction eluted with water (1) is incorporated as described previously, VY is able to be recovered in a large quantity than that of only peptide SY-MD and, in addition, a peptide mixture “peptide SY” having excellent taste and stability is able to be prepared.

All of peptides α-1000, Y-2, SY and SY-MD in the present invention are the substances derived from natural substances containing dipeptide VY, show excellent action for inhibition or suppression on calcium channel and, further, have no problem in terms of safety. Therefore, they may be also used as a peptide mixture for inhibitor or suppressor to calcium channel or as specific health food for the purpose of suppression as such. Accordingly, the present peptide mixtures are able to be used as additives to food or animal feed such as seasoning or food for enrichment of nutrition and, in addition, because of the above-mentioned unique physiological activity, they are able to be widely used for prevention or treatment of diseases of blood vessel as pharmaceutical agent, infusion, health food, food for clinical nutrition, etc.

In the present invention, the term reading inhibition of calcium channel widely means not only the case where calcium channel is completely inhibited but also the case of a partial inhibition or, in other words, suppression. Hereinafter, the term reading inhibition of calcium channel will be used in a sense including the above.

When the peptide mixture is used as a food, it may be appropriately used according to usual manner by adding as it is or using together with other food or food component(s). When it is used as a pharmaceutical agent, it may be administered either orally or parenterally. In the case of oral administration, it may be made into, for example, tablets, granules, powder, capsules, powder mixture or drink according to the usual method. In the case of parenteral administration, it may be used, for example, as injections, infusions and suppositories. It goes without saying that purified dipeptide VY may also be made into pharmaceuticals and foods (including beverages in the present invention) by the same manner as above.

### ADVANTAGES OF THE INVENTION

In accordance with the present invention, it has been firstly confirmed that VY inhibits calcium channel in normal human aortic vessel smooth muscle cells (VSMC) and a calcium channel inhibitor where VY is an effective ingredient has been developed. It has been further confirmed that a substance containing VY (such as peptide mixture derived from fish meat, sardine peptide mixture, peptide α-1000, Y-2, SY and SY-MD) is effective and a calcium channel inhibitor containing such a peptide mixture as an effective ingredient has been also developed for the first time.

In the present invention, an entirely novel action of a calcium channel inhibiting action of VY which is entirely different from an ACE inhibiting action and a novel development of the so-called second medical use has been succeeded. Further, in the present invention, in a hypotensive mechanism by VY, it has been firstly confirmed that there are two actions of ACE inhibition and calcium channel inhibition. Thus, it is really an epoch-making new finding and a new development such as development of hypotensive agent by a calcium channel inhibiting route is also able to be expected.

There is no problem in safety in all of VY and substances containing the same (such as the above-mentioned various peptide mixtures) (in fact, even when 500 mg/day was compulsorily administered orally to rats, there was no acute toxicity was observed after ten days.) and not only pharmaceutical effect but also taste are good. Therefore, they are able to be used as the inhibitor and, in addition, as foods such as peptides for specific health foods for the purpose of such an inhibition.

In addition, VY and substances containing the same have an excellent calcium channel inhibiting action and, therefore, they are able to be utilized as pharmaceuticals and food/beverage for prevention and/or treatment of cerebral infarction diseases, migraine diseases, epileptic diseases, mental disease, pain diseases, hypertension, angina pectoris, arrhythmia, cardiomyopathy, cerebral ischemia, cardiac insufficiency, ischemic coronary artery cardiac diseases, etc. and, further, their efficacy to suppression of gastralgia, reduction of winkles and slight wrinkles, prevention of arteriosclerosis, etc. are able to be well expected.

### EXAMPLES

Examples of the present invention will be mentioned as follows although the present invention is not limited thereto.

### Example 1

### Manufacture of Peptide α-1000

Fresh sardines were processed in a deboner to collect the meat. The meat was ground and divided into plural lumps of ground fish meat weighing 10 kg each, and these meat lumps were rapidly frozen at a temperature of not higher than −30° C. Then each meat lump was milled in a mill, to which was added water of the same amount as that of the meat. The resulting mixture was fed into a tank, then heated therein at 100° C. for 10 minutes whereby the autolytic enzyme in the meat was inactivated and the meat was thermally denatured. Next, aqueous ammonia was added to this, with which the pH-value of the processed meat was adjusted to 9.5.

A 0.1 w/v % solution of a commercially-available alkaline protease was added thereto. Then the resulting meat was kept heated at 50° C. for 17.5 hours so as to be decomposed with the enzyme added thereto. Next, this was boiled for 15 minutes to inactivate the enzyme used.

This was then passed through a vibro-screen (150 meshes) and then treated by Jector (at 5,000 rpm) and thereafter processed in a Sharples type centrifugal separator (at 15,000 rpm). Then this was filtered using diatomaceous earth as a filter aid and the resulting filtrate was used as a solution containing peptides.

Activated carbon was added to the obtained unpurified solution in an amount of 1 w/v %, then stirred at 30° C. for 60 minutes, and thereafter filtered. The filtrate was concentrated under reduced pressure (at 20° C.) in an ordinary manner and then sterilized through UHTST also in an ordinary manner to obtain a product peptide α-1000 (liquid). This was further spray-dried in an ordinary manner into a product peptide α-1000 (powdery) having a particle size of 60 meshes. Each of these products was frozen and stored.

### Example 2

### Manufacture of Peptide Y-2 (1)

Sardine meat was subjected to a decomposing process with 0.7 w/v % alkalase for 17.5 hours, the resulting hydrolysate was applied to an ODS column, the latter half part of the fraction eluted with water and the fraction eluted with a 15 v/v % aqueous ethanol solution conducted thereafter were mixed and the resulting fraction mixture was used as peptide Y-2. Peptide Y-2 contains VY of 150 mg/100 g of the resulting fraction.

### Example 3

### Manufacture of Peptide Y-2 (2)

Deionized water (26.2 liters) was added to 800 ml of the sardine peptide α-1000 (liquid) prepared in Example 1 (Brix 45, having a protein content of 29.6 w/v %) and applied to a column (1.5×50 cm) filled with ODS resin (YMC ODS-AQ 120-S50) in which peptides contained therein were adsorbed onto the resin. Then, the column was washed with deionized water and eluted with, 0 v/v %, 10 v/v %, 25 v/v %, 50 v/v % and 99.5 v/v % aqueous ethanol solutions of 27 liters each in that order to obtain fractions of Y-1, Y-2, Y-3, Y-4 and Y-5, respectively. Of those, Y-2 fraction was concentrated at 40° C. to remove ethanol therefrom and then freeze-dried to obtain a purified sardine peptide mixture (Y-2). The Y-2 fraction contained about 2- to 3-fold of VY as compared with peptide α-1000.

### Example 4

### Manufacture of Peptide SY

5 g of sardine peptide α-1000 (powdery) prepared in Example 1 was dissolved in 500 ml of deionized water to give a unpurified solution, applied into a column (3.5×13 cm) of a hydrophobic adsorptive resin SEPABEADS SP 207 (manufactured by Mitsubishi Chemical Co.) so as to fill the column with the prepared α-1000 solution (unpurified solution application) and, according to the eluting pattern of FIG. 11, each 500 ml of water, a 15 v/v % aqueous solution of ethanol and water were added so that all the fractions of the same sardine peptide SY as shown in FIG. 11 or, in other words, a latter fraction eluted with water (1), a fraction with a 15 v/v % ethanol solution and a fraction eluted with water (2) were collected, mixed and freeze-dried whereupon 2.1 g of peptide SY (powdery) was prepared. The Na content in peptide SY was 1.45 w/w % (according to an atomic absorption spectrophotometry).

### Example 5

### Manufacture of Peptide SY-MD

5 g of sardine peptide α-1000 (powdery) obtained in Example 1 was dissolved in 500 ml of deionized water to give a unpurified solution, applied into a column (3.5×13 cm) of a hydrophobic adsorptive resin SEPABEADS SP 207 (manufactured by Mitsubishi Chemical Co.) so as to fill the column with the prepared α-1000 solution (unpurified solution application) and only a fraction eluted with a 15 v/v % ethanol solution among all the fractions of the same sardine peptide SY as shown in the eluting pattern of FIG. 11 was isolated and collected followed by freeze-drying to give 1.7 g of peptide SY-MD (powdery). The Na content of peptide SY-MD was 0.124 w/w % (according to an atomic absorption spectrophotometry).

### Example 6

### Manufacture of Drinks

Table for Formulation (per/100 ml) for a 100-ml Drink

Liquid sugar comprising fructose and glucose

| | | |
|---|---|---|
| Liquid sugar comprising fructose and glucose | 4.5 | g |
| Sugar alcohol | 1 | g |
| Acidifier | 0.2 | g |
| Flavoring | 0.13 | g |
| Sweetener (Stevia) | 0.03 | g |
| Caramel dye | 0.02 | g |
| Peptide SY (powdery) (prepared in Example 4) | 0.5 | g |

Pure water was added to make the total volume 100 ml.

Table for Formulation (per 50 ml) for a 50-ml Drink

Liquid sugar comprising fructose and glucose

| | | |
|---|---|---|
| Liquid sugar comprising fructose and glucose | 10 | g |
| Flavoring | 0.3 | g |
| Acidifier | 0.16 | g |
| Sweetener (Stevia) | 0.015 | g |
| Peptide SY (powdery) (prepared in Example 4) | 0.5 | g |

Pure water was added to make the total volume 50 ml.

Table for Formulation (per 30 ml) for a 30-ml Drink

Liquid sugar comprising fructose and glucose

| | | |
|---|---|---|
| Liquid sugar comprising fructose and glucose | 5 | g |
| Flavoring | 0.25 | g |
| Acidifier | 0.1 | g |
| Sweetener (Stevia) | 0.015 | g |
| Peptide SY (powdery) (prepared in Example 4) | 0.5 | g |

Pure water was added to make the total volume 30 ml.

The ingredients for each drink were mixed, respectively, and dissolved at 60° C., and subjected to plate sterilization at 128° C. for 10 seconds. After that, the respective mixtures were filled, at 90° C. into each of 100-ml, 50-ml and 30-ml brown bottles having been well washed, and left cooled at room temperature and then rapidly cooled with running water in a bath, thereby each drink being produced.

### Example 7

### Manufacture of Tablets

Tablets were manufactured according to the following formulation.

500 g of peptide SY (powdery) prepared by the same method as in Example 4, 356 g of reduced maltose syrup, 100 g of crystalline cellulose, 40 g of sucrose fatty acid ester and 4 g of a sweetener (stevia) were mixed and the mixture was compressed using a compressive tabletting machine to prepare core tablets (4,000 tablets×250 mg). The core tablets were coated with 7.5 mg of shellac per tablet to manufacture 4,000 tablets containing 500 mg of peptide SY (powdery) per 4 tablets.

In accordance with the same manner as above, drinks and tablets were manufactured using peptide α-1000 manufactured by the same method as in Example 1, peptide Y-2 manufactured by the same method as in Examples 2 and 3 and peptide SY-MD manufactured by the same method as in Example 5.

### Example 8

### Test for Calcium Channel Inhibition

As to cells, normal human aortic vessel smooth muscle cells (VSMC; Cyro AOSMC (trade name) manufactured by Sanko Junyaku Co.) were used while, as to VY, valyl-tyrosine which was chemically synthesized was used and a calcium channel inhibiting action by VY was confirmed.

i) Influence of VY on VSMC

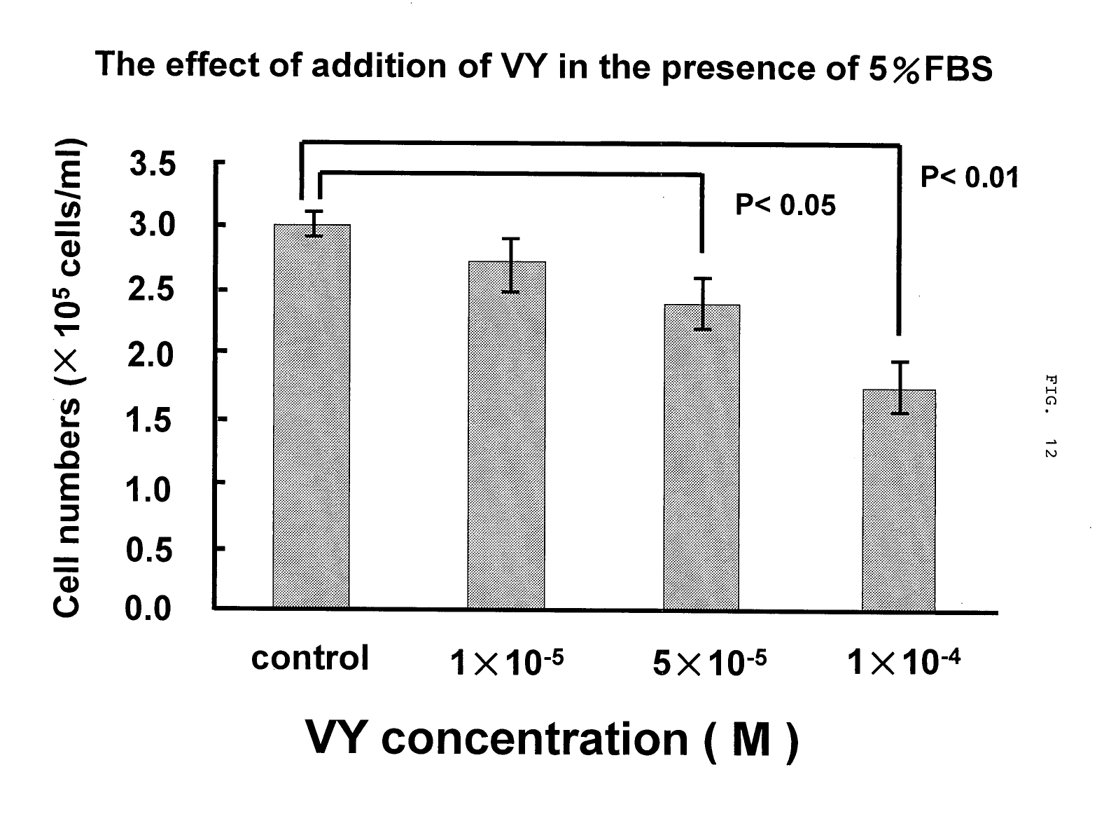

VSMC (1×10⁵ cell/ml) was pre-incubated for 24 hours in a serum-free medium and transferred to a medium containing 5 v/v % of FBS (fetal bovine serum), 1 w/v % of hEGF (human epidermal growth factor) and 1 w/v % of hEGF-β, then VY was added thereto so as to make its concentrations 0, 50 and 100 μM and incubation of the resulting mixtures was conducted for 5 days at 37° C. in a CO₂ incubator. This was stained with Trypan Blue and living cells were counted by a blood corpuscle counter. The result which is made into a graph is FIG. 12 (effect of addition of VY in the presence of 5% FBS). As a result, it was found that cell growth was suppressed in proportion to the added VY concentration.

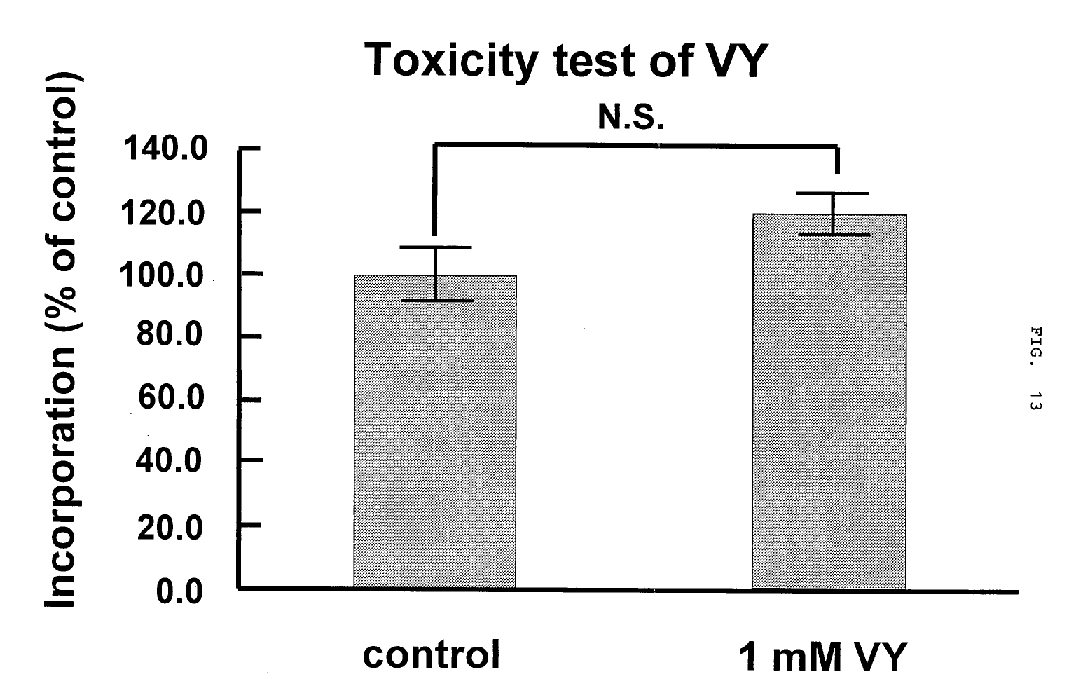

In order to check the toxicity of VY, VSMC was pre-incubated for 24 hours on a serum-free medium on a 96-well plate, 0 mM or 1 mM of VY was added thereto and incubation was conducted for 48 hours. To this was added a solution containing 10 μM of WST-8 (2-(2-methoxy-4-nitrophenyl)-3-(4-nitrophenyl)-5-(2,4-disulfophenyl)-2H-tetrazolium (Cell Counting Kit-8; manufactured by Dojin Kagaku CO.)), color reaction was conducted in a CO₂ incubator at 37° C. for 3 hours and the amount of the resulting water-soluble formazan was determined by absorbance at 450 nm where cell growth was measured from the synthesized amount of DNA. The result which was made into a graph is FIG. 13 (VY toxicity test). As a result, it was noted that there was no difference between their cell growths and that VY is non-toxic.

ii) Influence of VY on Ang II Stimulation

When Ang II (angiotensin II) is added to an incubated liquid of VSMC, cell growth thereof is promoted. It has been known to be due to the fact that Ang II is bonded to an AT1 receptor existing on the surface of VSMC, the bonding signal flows in the cells and, as a result, calcium channel opens and calcium ion flows into the cells. Such a mechanism was utilized to conduct the following experiment.

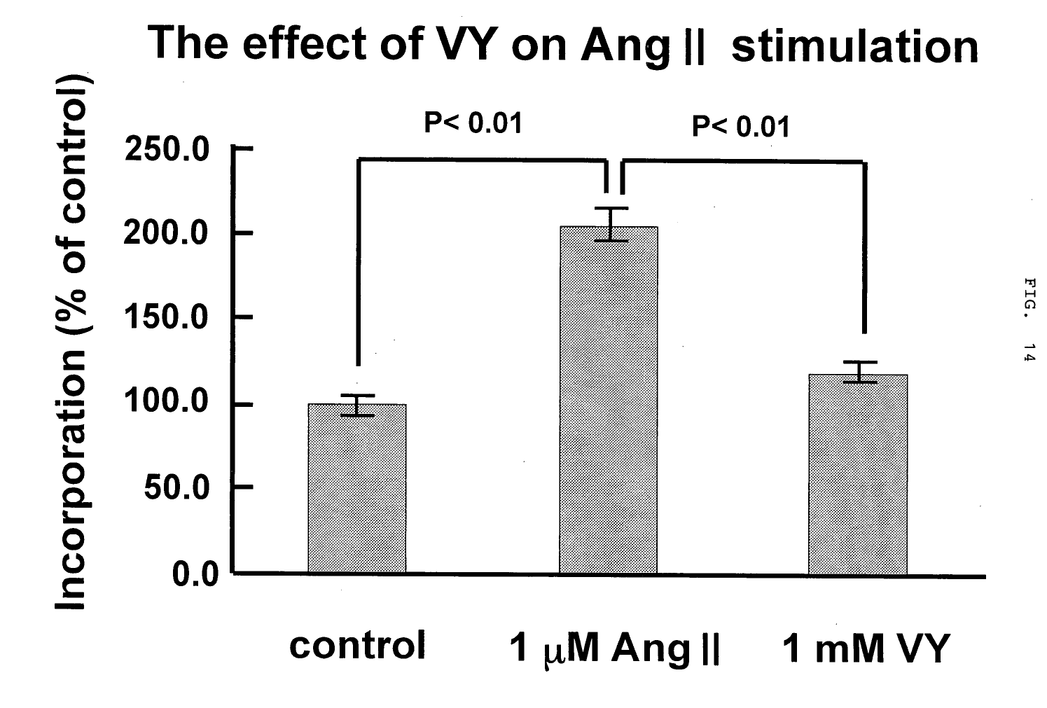

VSMC was pre-incubated for 24 hours on a serum-free medium on a 96-well plate and three kinds of samples—a control to which no additive was added, a sample to which only Ang II was added in a concentration of 1 μM (Ang II (+)) and a sample to which Ang II and VY were added in concentrations of 1 μM and 1 mM, respectively (Ang II(+)VY(+))—were incubated for 48 hours. Then 10 μM of WST-8 was added thereto and amount of water-soluble formazan resulted by color reaction at 37° C. for 3 hours in a CO₂ incubator was measured from absorbance at 450 nm whereupon cell growth was measured form the synthesized amount of DNA. The result which was made into a graph is FIG. 14 (effect of VY on Ang II stimulation). As a result, it was noted that growth was strongly promoted by Ang II(+) while cell growth was not promoted by Ang II(+)VY(+) whereby VY had a suppressive effect to VSMC growth by Ang II stimulation.

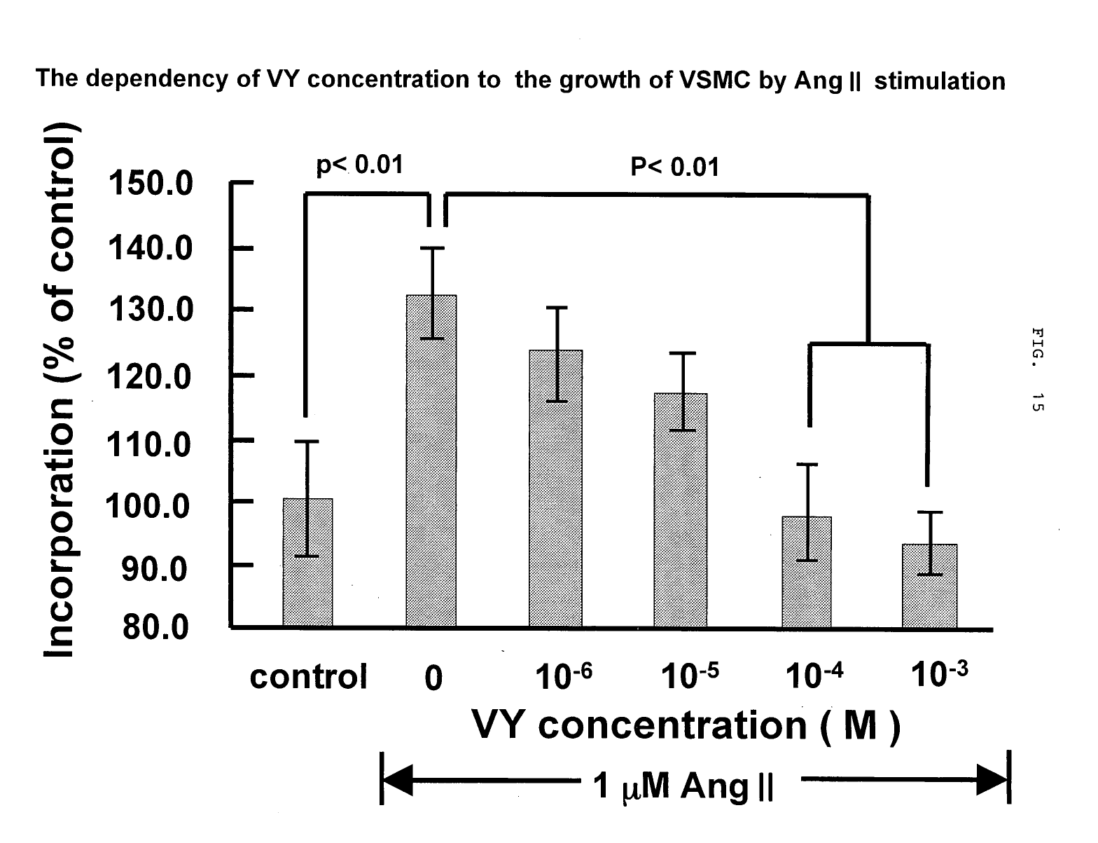

Further, cell growth when concentration of VY added by the same method was made 0 μM, 1 μM, 10 μM, 100 μM and 1 mM was measured. That is, VSMC was pre-incubated on a serum-free medium for 24 hours on a 96-well plate and six kinds of samples—a control where nothing was added and five samples where Ang II was added in a concentration of 1 μM and VY was further added in concentrations 0 μM, 1 μM, 10 μM, 100 μM and 1 mM—were incubated for 48 hours. Then 10 μM WST-8 was added thereto and amount of water-soluble formazan produced by color reaction at 37° C. for 3 hours in a CO₂ incubator was determined from absorbance at 450 nm whereupon cell growth was measured from the synthesized amount of DNA. The result which was made into a graph is FIG. 15 (dependency of VY concentration to VSMC growth by Ang II stimulation). As a result, it was noted that VY had a suppressive effect to the growth of VSMC by Ang II stimulation in a concentration-depending manner.

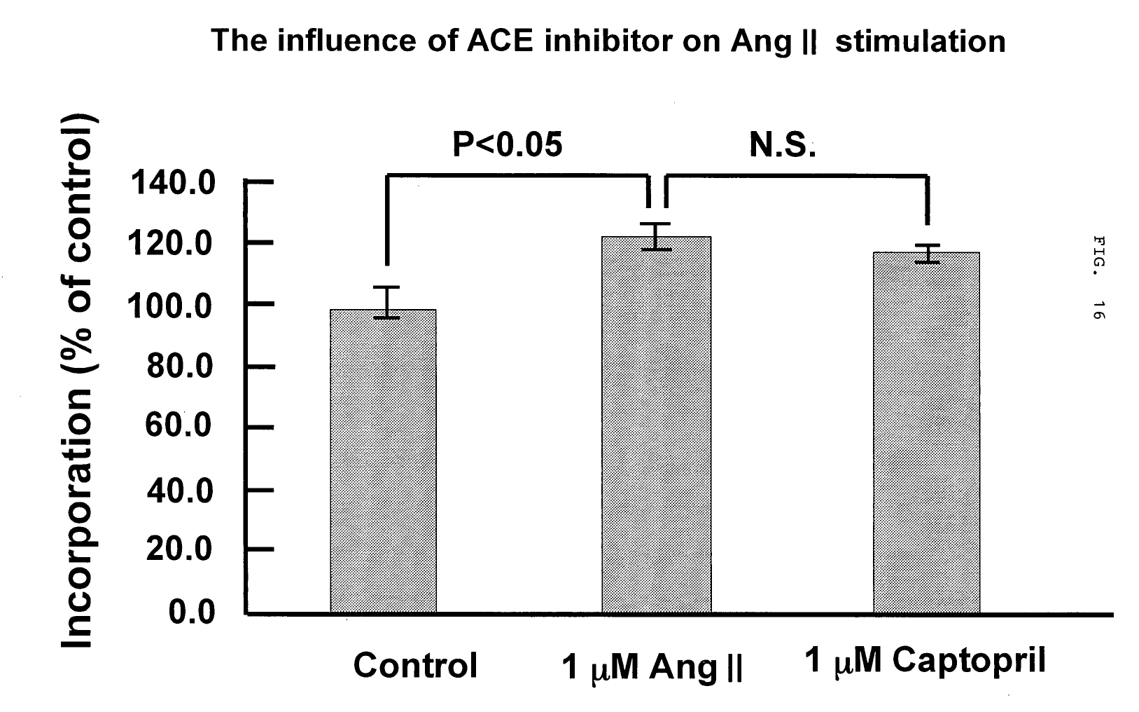

VSMC was pre-incubated in a serum-free-medium for 24 hours on a 96-well plate, then 1 μM of captopril which is an ACE inhibitor was added thereto and incubation was conducted for 48 hours. Then 10 μM WST-8 was added thereto and amount of water-soluble formazan produced by color reaction for 3 hours was determined from absorbance at 450 nm whereupon cell growth was measured from synthesized amount of DNA. The result which was made into a graph is FIG. 16 (influence of ACE inhibitor on Ang II stimulation). As a result, captopril did not show a suppressive action to Ang II stimulation.

From those results, it was clarified that suppressive effect to Ang II stimulation by VY was not due to an ACE inhibitive activity.

iii) Influence of VY on Ang II Receptor

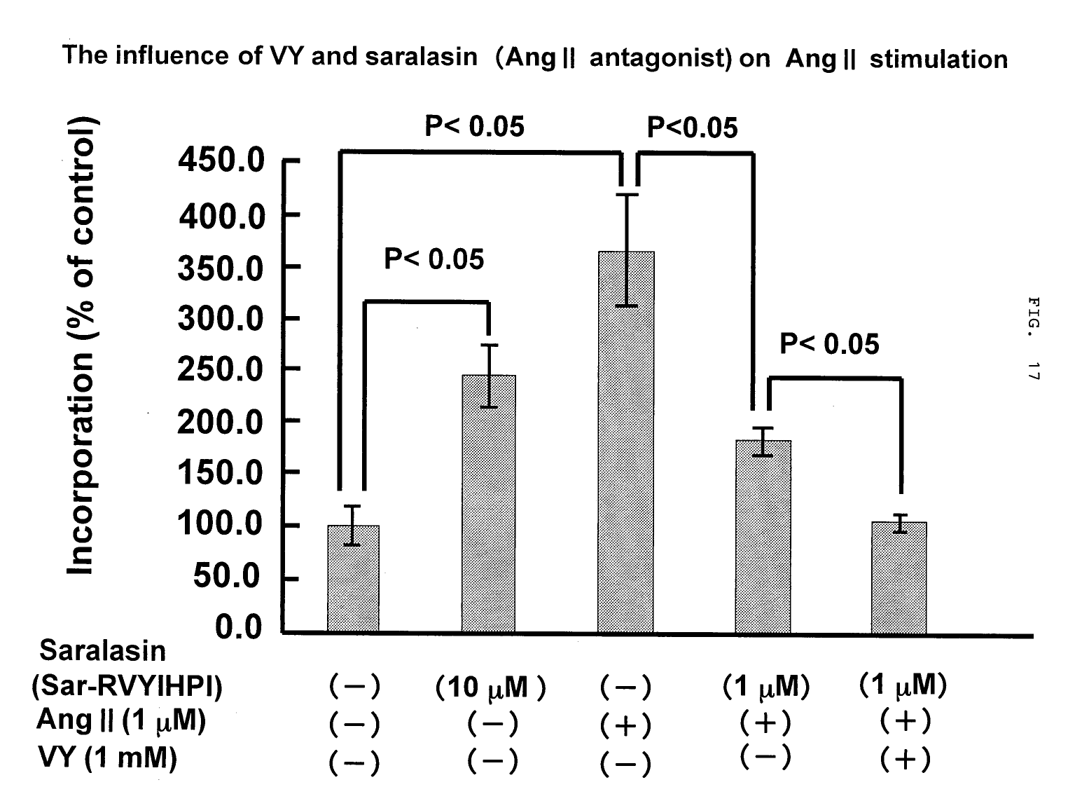

Influence of VY on Ang II receptor AT1 existing on the surface of VSMC was investigated. That is, VSMC was pre-incubated in a serum-free medium for 24 hours on a 96-well plate and incubation was conducted for 48 hours after 1 μM of Ang II was added, 10 μM of saralasin which is an Ang II antagonist was added, each 1 μM of Ang II and saralasin were added or each 1 μM of Ang II and saralasin and 1 mM of VY were added. Then 10 μM of WST-8 was added thereto and amount of water-soluble formazan formed by color reaction for 3 hours at 37° C. in a CO₂ incubator was determined by absorbance at 450 nm whereby cell growth was measured from the synthesized amount of DNA. The result which was made into a graph is FIG. 17 (effect of VY and saralasin (Ang II antagonist) on Ang II stimulation). As a result, even when saralasin was added, cell growth was promoted although that was not so significant as in the case of addition of Ang II and, when both Ang II and saralasin were added, saralasin shows a competitive inhibitory action whereupon the growth promotion effect was weak as compared with the case of sole use of saralasin. It was also noted that, when Ang II, saralasin and VY were added to the incubated liquid, no growth promoting effect was noted and accordingly that suppressive action of VY to Ang II stimulation was not due to inhibition action of Ang II on the receptor.

iv) Influence of VY on Calcium Channel Agonist Stimulation

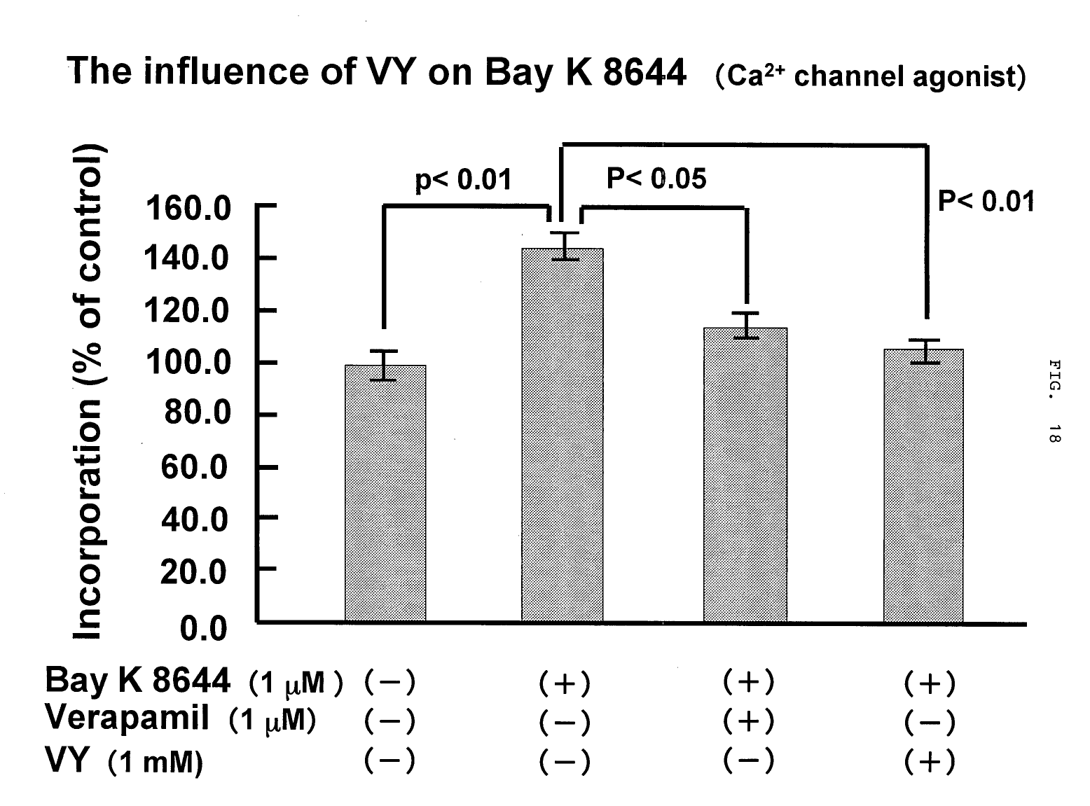

Inflow of calcium ion which is downstream of Ang II stimulation showing a cell growth promoting action was investigated. That is, VSMC was pre-incubated in a serum-free medium for 24 hours on a 96-well plate, and incubation was conducted for 48 hours after addition of 1 μM of Bay K 8644 which is a calcium channel agonist, addition of each 1 μM of Bay K 8644 and verapamil which is a calcium channel inhibitor or addition of 1 μM of Bay K 8644 and 1 mM of VY. Then 10 μM of WST-8 was added thereto and amount of water-soluble formazan produced by color reaction for 3 hours at 37° C. in a CO₂ incubator was determined by absorbance at 450 nm whereupon cell growth was measured from the synthesized amount of DNA. The result which was made into a graph is FIG. 18 (influence of VY on Bay K 8644 (Ca²⁺ channel agonist, Sigma Co.) stimulation). As a result, it was noted that cell growth was promoted by Bay K 8644, that addition of Bay K 8644 and verapamil suppressed the cell growth promoting activity and that addition of Bay K 8644 and VY suppressed cell growth promoting activity as same as in the case of addition of verapamil.

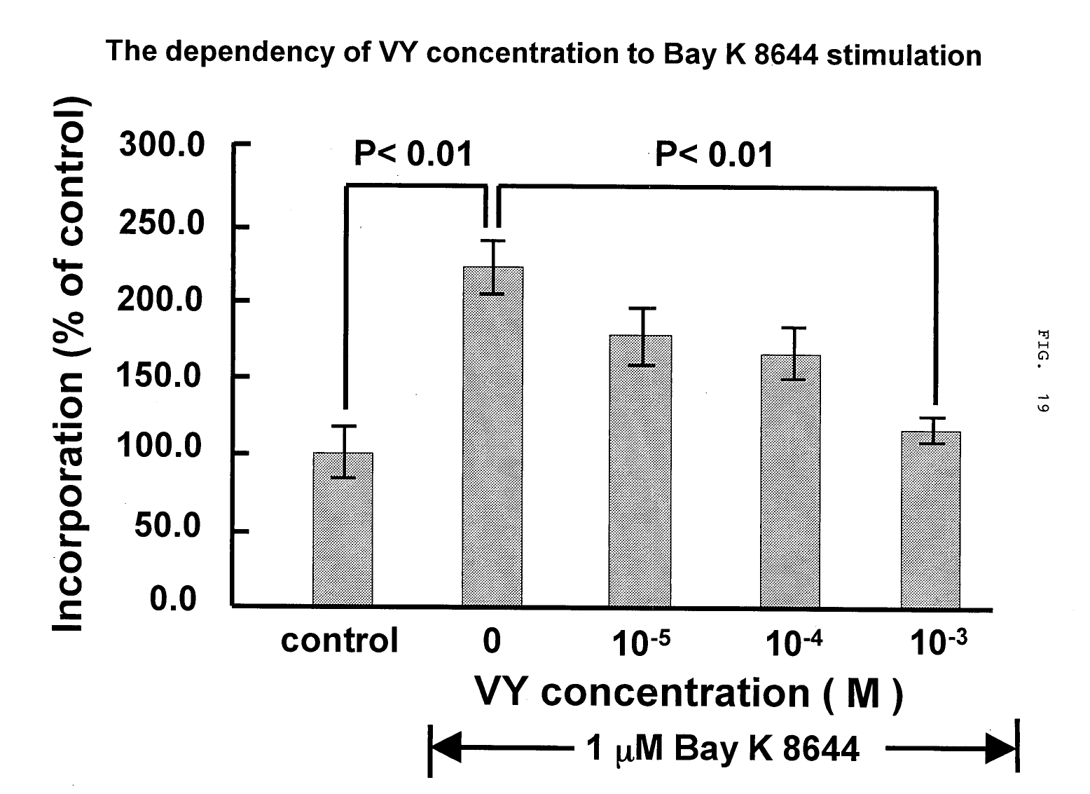

Further, cell growth where concentration of VY which was added together with 1 μM of Bay K 8644 in the same manner was made 0, 10 μM, 100 μM and 1 mM was measured. That is, VSMC was pre-incubated for 24 hours in a serum-free medium on a 96-well plate, and incubation was conducted for 48 hours after addition of nothing as a control and after addition of 1 μM of Bay K 8644 together with 0, 10 μM, 100 μM or 1 mM of VY. Then 10 μM of WST-8 was added thereto and amount of water-soluble formazan produced by color reaction for 3 hours at 37° C. in a CO₂ incubator was determined by absorbance at 450 nm whereby cell growth was measured from synthesized amount of DNA. The result which was made into a graph is FIG. 19 (dependency on concentration of VY to Bay K 8644 stimulation). As a result, it was noted that VY is a calcium channel inhibitor having a suppressive effect to VSMC growth by Bay K 8644 stimulation in a concentration-dependent manner.

### v) CONCLUSION

Consequently, it was clarified that a suppressive effect of VY to VSMC growth by Ang II stimulation was due to a calcium channel inhibition.

1. A calcium channel inhibitor, wherein dipeptide Val-Tyr or a substance containing dipeptide Val-Tyr is an effective ingredient.

2. The calcium channel inhibitor according to claim 1, wherein the substance containing dipeptide Val-Tyr is a peptide mixture derived from fish meat.

3. The calcium channel inhibitor according to claim 2, wherein the peptide mixture derived from fish meat is at least one member selected from the group consisting of peptides α-1000, Y-2, SY and SY-DM as shown in the following (a) to (d):
(a) a peptide α-1000 which is prepared in such a manner that fish meat is thermally denatured and hydrolyzed by treating with neutral or alkaline protease, the enzyme is inactivated and a separating treatment is conducted;
(b) a peptide Y-2 comprising a fraction which is prepared in such a manner that an aqueous solution of the peptide α-1000 is used as a unpurified solution containing peptide, applied to a peptide-adsorbing resin and eluted with a 8 to 17 v/v % aqueous solution of ethanol;
(c) a peptide SY which is prepared in such a manner that the unpurified solution containing peptide in the above (b) is applied to a peptide-adsorbing resin and eluted with water (1), aqueous solution of ethanol and water (2) in this order, and the eluted three fractions are mixed, which three fractions are apart of a fraction eluted with water (1), a fraction eluted with the aqueous solution of ethanol and a fraction eluted with water (2); and
(d) a peptide SY-MD which is the same fraction eluted with the same aqueous solution of ethanol as in the above (b).

4. A calcium channel inhibitor, wherein an effective ingredient is a peptide α-1000 containing dipeptide Val-Tyr which is prepared in such a manner that fish meat is thermally denatured and hydrolyzed by treating with neutral or alkaline protease, the enzyme is inactivated and a separating treatment is conducted.

5. A calcium channel inhibitor, wherein a peptide α-1000 having the following physico-chemical properties is an effective ingredient:
(A) Molecular weight: 200 to 10,000 (as measured by Sephadex G-25 column chromatography);
(B) Melting point: Colored at 119° C. (decomposition point);
(C) Specific rotatory power: [α]_{D}²⁰=−22°;
(D) Solubility in solvents: It is easily soluble in water but rarely soluble in ethanol, acetone and hexane;
(E) Chemical differentiation in acidic, neutral or basic character: Neutral;
(F) Appearance and constituent components: It is white powder comprising 5.14 w/w % of water (by a vacuum heating and drying method), 87.5 w/w % of protein (by a Kjeldahl method with a nitrogen/protein conversion coefficient of 6.25), 0 w/w % of lipid (by a Soxhlet extraction method) and 5.0 w/w % of ash (by a direct ashing method);
(G) Characteristics: It is a peptide mixture derived from fish meat and obtained by inactivating an autolytic enzyme by heating followed by hydrolyzing with protease; it contains dipeptide Val-Tyr and has an action of inhibition or suppression of calcium channel;
(H) Infrared absorption spectrum: FIG. 1; and
(I) Ultraviolet absorption spectrum: FIG. 2.

6. A calcium channel inhibitor, wherein an effective ingredient is a peptide Y-2 containing dipeptide Val-Tyr which is prepared in such a manner that the aqueous solution of the peptide α-1000 mentioned in claim 4 is used as a unpurified solution containing peptide, applied to a peptide-adsorbing resin and eluted with a 8 to 17 v/v % aqueous solution of ethanol.

7. A calcium channel inhibitor, wherein a peptide Y-2 having the following physico-chemical properties is an effective ingredient:
(A) Molecular weight: 200 to 10,000 (as measured by high-performance liquid chromatography using ASAHIPAK GS-320);
(B) Melting point: It is colored and decomposed at 138° C.;
(C) Specific rotatory power: [α]_{D}²⁰=−40°;
(D) Solubility in solvents: It is easily soluble in water but rarely soluble in ethanol, acetone and hexane;
(E) Chemical differentiation in acidic, neutral or basic character: Neutral; pH of from 5.0 to 8.0 (10 w/v % solution);
(F) Appearance of the substance: It is white to light yellow powder;
(G) Constituent components: It comprises 2.72 w/w % of water (by a heating and drying method under ordinary pressure), 87.25 w/w % of protein (by a Kjeldahl method with a nitrogen/protein conversion coefficient of 6.25), 0 w/w % of lipid (by a Soxhlet extraction method) and 0.20 w/w % of ash (by a direct ashing method);
(H) Physiological properties: It contains a dipeptide Val-Tyr and has an inhibitive or suppressing action to calcium channel;
(I) Infrared absorption spectrum: FIG. 4; and
(J) Ultraviolet absorption spectrum: FIG. 5.

8. A calcium channel inhibitor, wherein an effective ingredient is a peptide SY containing dipeptide Val-Tyr which is a peptide mixture prepared by applying a unpurified solution containing peptide (the aqueous solution of the peptide α-1000 mentioned in claim 4) into a column of a peptide-adsorbing resin followed by subjecting to an elution and fractionation treatment and which is conducted in such a manner that, in the same eluted patterns as shown in FIG. 11 which are obtained by the fractionation treatment by elution in the order of water, an aqueous solution of ethanol and water as eluents, a latter fraction by eluting with water (1), a fraction by eluting with a 11 to 18 v/v % ethanol and a fraction eluted with water (2) obtained by each eluent as stipulated below are prepared and mixed to produce a peptide SY:
(1) Latter fraction by eluting with water (1): A fraction obtained by using water as an eluent of from a time when a sodium (Na) content of the whole fraction (peptide SY) eluted becomes 1 to 3 g/100 g to a final collection time of the latter fraction in the water elution (1) when the sodium content becomes substantially 0 g/100 g;
(2) Fraction by eluting with ethanol: A fraction obtained next by using the ethanol aqueous solution having a concentration of 11 to 18 v/v % as an eluent until an amount of peptide eluted passes a peak and decreases to about one-half of the peak; and
(3) Fraction by eluting with water (2): A fraction thereafter obtained by using water as an eluent until the elution of peptide is completed.

9. A calcium channel inhibitor, wherein a peptide SY having the following physico-chemical properties is an effective ingredient:
(A) Molecular weight: 200 to 10,000 (as measured by high-performance liquid chromatography using ASAHIPAK GS-320);
(B) Melting point: It is colored and decomposed at 138 ±3° C.;
(C) Solubility in solvents: It is easily soluble in water but rarely soluble in ethanol, acetone and hexane;
(D) Appearance and property: It is white to light yellow powder;
(E) Liquid property (pH): 4.0 to 6.0;
(F) Constituent components: It comprises 1 to 5 w/w % of water (by a heating and drying method under ordinary pressure), 84 to 94 w/w % of protein (by a micro-Kjeldahl method), 0.5 w/w % of lipid (by a Soxhlet extraction method), 4±2 w/w % of ash (by a direct ashing method) and 1 to 3 w/w % of Na (by an atomic absorption spectrophotometry);
(G) Physiological properties: It contains a dipeptide Val-Tyr and has an inhibitive or suppressive action to calcium channel;
(H) Infrared absorption spectrum: FIG. 7; and
(I) Ultraviolet absorption spectrum: FIG. 8.

10. A calcium channel inhibitor, wherein an effective ingredient is a peptide SY-MD containing dipeptide Val-Tyr which is a fraction obtained until an amount of peptide eluted by using the aqueous solution of ethanol as an eluent in (2) of claim 8 passes the peak and decreases to about one-half of the peak is isolated and collected.

11. A calcium channel inhibitor, wherein a peptide SY-MD having the following physico-chemical properties is an effective ingredient:
(A) Molecular weight: 200 to 10,000;
(B) Melting point: It is colored and decomposed at 138 ±3° C.;
(C) Solubility in solvents: It is easily soluble in water but rarely soluble in ethanol, acetone and hexane;
(D) Appearance and property: It is white to light yellow powder;
(E) Liquid property (pH): 4.0 to 6.0;
(F) Constituent components: It comprises 2 to 6 w/w % of water (by a heating and drying method under ordinary pressure), 90 to 98 w/w % of protein (by a micro-Kjeldahl method), 0.5 w/w % of lipid (by a Soxhlet extraction method), 3.0 w/w % of ash (by a direct ashing method) and 0.1 to 0.2 w/w % of Na (by an atomic absorption spectrophotometry);
(G) Physiological properties: It contains a dipeptide Val-Tyr and has an inhibitive or suppressive action to calcium channel;
(H) Infrared absorption spectrum: FIG. 9; and
(I) Ultraviolet absorption spectrum: FIG. 10.

12. Food for inhibition or suppression of calcium channel, wherein a substance which is derived from natural substance and contains dipeptide Val-Tyr is an effective ingredient.

13. Food for inhibition or suppression of calcium channel, which comprises the peptide α-1000 mentioned in claim 5.

14. Food for inhibition or suppression of calcium channel, which comprises the peptide Y-2 mentioned in claim 7.

15. Food for inhibition or suppression of calcium channel, which comprises the peptide SY mentioned in claim 9.

16. Food for inhibition or suppression of calcium channel, which comprises the peptide SY-MD mentioned in claim 11.

17. A method for inhibiting calcium channel in a human in need thereof, which comprises administering a material comprising dipeptide Val-Tyr to the human in an effective amount.

18. A method for inhibiting calcium channel in a human in need thereof, which comprises ingesting a food comprising dipeptide Val-Tyr in an effective amount.
